(19) United States
(12) Patent Application Publication
(10) Pub. No.: US 2011/0076757 A1
(43) Pub. Date: Mar. 31, 2011

(54) AUTOMATED ALGAE CULTURE APPARATUS

(75) Inventors: Yung-Hsiang Yang, Taipei City (TW); Ming-Chen Yu, Taipei City (TW); Jiunn-Ming Jeng, Taipei County (TW); Walter Dan, Taichung City (TW)

(73) Assignee: BYOTEC BIOTECHNOLOGY CO. LTD, Miaoli County (TW)

(21) Appl. No.: 12/889,418

(22) Filed: Sep. 24, 2010

(30) Foreign Application Priority Data
Sep. 25, 2009 (TW) 98217748

(51) Int. Cl. C12M 1/12

(52) U.S. Cl. 435/292.1

(57) ABSTRACT

An automated algae culture apparatus includes at least one photosynthetic reactor module and an auxiliary equipment module. The photosynthetic reactor module includes a transparent container used to contain an algae solution which includes algae. The auxiliary equipment module includes a hydraulic filter, a buffer tank and an aerator. The hydraulic filter is used to filter the algae in the algae solution, and is communicated with a water outlet of the transparent container to filter the algae by gravity force. The buffer tank is communicated with a water inlet of the transparent container. The aerator is communicated with an air inlet of the transparent container.

### CROSS-REFERENCE TO RELATED APPLICATION

This application claims the priority benefit of Taiwan application serial No. 98217748, filed on Sep. 25, 2009. The entirety of the above-mentioned patent application is hereby incorporated by reference herein and made a part of this specification.

### BACKGROUND OF THE INVENTION

1. Field of the Invention

The invention is related to an algae culture apparatus, use thereof, and an algae culture method, and particularly to an automated algae culture apparatus, use thereof, and an automated algae culture method.

2. Description of Related Art

The use of culture equipment to culture algae and absorb carbon dioxide through photosynthesis to reduce carbon emissions has been known to persons in the art. Efficiencies of carbon fixation by microalgae may reach tens of times of that by land plants, thereby making algae the best example in using living organisms for reducing carbon emissions.

Currently, algae culture equipment, such as that used in large-scale farms, utilize open horizontal pools, so that large areas are required and heavy influences by the weather are present. Yields are therefore unstable, and there is also the danger of external bio-contamination. In order to overcome these problems, so-called sealed photosynthetic bioreactors are produced. In the so-called photosynthetic bioreactors, a culture medium is filled therein, and after inoculating the algae, carbon dioxide is provided by an aerating method, and the algae is cycled in the photosynthetic bioreactor.

When the algae grows to a certain concentration, by using a continuous centrifuge, press filter, and spray dryer, the algae is dried into dried algae for further processing.

This type of photosynthetic bioreactors is mostly used on algae farms or in professional laboratories, and multiple auxiliary equipments, such as aerators, nutrient supplement tanks, water supply pumps, algae recycle equipment, and professional operators, are required for operation and maintenance. Hence these photosynthetic bioreactors may only be used in professional commercial operations and are hard to popularize.

In addition, in current days when global warming is serious, if algae culture equipment is popularized so that algae is easily cultured in public space or households, just as in fishtanks common in most households, it would be beneficial to further enlarging the scale of saving power and reducing carbon emissions. In addition, in public spaces crowed with people, culturing microalgae rapidly and effectively purifies the indoor air. The above are all advantages that cannot be achieved by current professional culture equipment.

There are two main technical obstacles that are required to be overcome. One is how to easily and effectively harvest microalgae, so that the biomass of algae in the culture equipment is maintained at log phase and reaches the greatest density. The other is how to maintain, in the sealed culture system, stability of the environment, for example maintaining parameters such water quality, pH value, nutrient contents, lighting conditions, and aeration at levels suitable for algae growth for long periods of time.

### SUMMARY OF THE INVENTION

In light of the above, an objective of the invention is to provide an automated algae culture apparatus, so as to miniaturize and popularize algae culture equipment.

The invention provides an automated algae culture apparatus which includes at least one photosynthetic reactor module and an auxiliary equipment module. The photosynthetic reactor module includes a transparent container used to contain an algae solution which includes algae. The auxiliary equipment module includes a hydraulic filter, a buffer tank and an aerator. The hydraulic filter is used to filter the algae in the algae solution and is communicated with a water outlet of the transparent container to filter the algae by gravity force. The buffer tank is communicated with a water inlet of the transparent container. The aerator is communicated with an air inlet of the transparent container.

According to an embodiment of the invention, in the automated algae culture apparatus, the photosynthetic reactor module further includes an auxiliary light source which is disposed on the transparent container.

According to an embodiment of the invention, in the automated algae culture apparatus, the hydraulic filter includes a filter bag or filter screen.

According to an embodiment of the invention, in the automated algae culture apparatus, when the automated algae culture apparatus includes a plurality of photosynthetic reactor modules, an arrangement of the photosynthetic reactor modules includes a parallel arrangement, a serial arrangement, or a matrix arrangement.

According to an embodiment of the invention, in the automated algae culture apparatus, the hydraulic filter is communicated with the buffer tank.

According to an embodiment of the invention, in the automated algae culture apparatus, the hydraulic filter is communicated with the aerator.

According to an embodiment of the invention, in the automated algae culture apparatus, the auxiliary equipment module further includes a fluid level restoring pump which is communicated with the transparent container and the buffer tank.

According to an embodiment of the invention, in the automated algae culture apparatus, the auxiliary equipment module further includes a central controller which is used to monitor an environmental parameter of the algae solution.

According to an embodiment of the invention, in the automated algae culture apparatus, the environmental parameter includes a temperature of the algae solution, a pH value of the algae solution, a fluid level of the algae solution, a first carbon dioxide concentration at a gas inlet of the transparent container, the second carbon dioxide concentration at a gas outlet of the transparent container, a lighting time, a harvest time, or any combination of the above parameters.

According to an embodiment of the invention, the automated algae culture apparatus further includes an anti-fogging filter module which covers the gas outlet of the transparent container, and the anti-fogging filter module includes a filter screen and an anti-fogging partition. The filter screen is disposed above the gas outlet. The anti-fogging partition is disposed between the gas outlet and the filter screen.

According to an embodiment of the invention, in the automated algae culture apparatus, the transparent container includes a thin-plate container or a columnar container.

According to an embodiment of the invention, in the automated algae culture apparatus, depending on whether the photosynthetic reactor module and the auxiliary equipment module are disposed in separation or in combination, the automated algae culture apparatus includes a separate apparatus or a combined apparatus.

According to an embodiment of the invention, in the automated algae culture apparatus, the auxiliary equipment module corresponds to one or more photosynthetic reactor modules.

Due to the above, since the automated algae culture apparatus in the invention integrates a plurality of components into the auxiliary equipment module, the algae culture equipment is miniaturized and popularized.

In addition, by using the automated algae culture apparatus, the algae is automatically recycled by the central controller and the hydraulic filter. Moreover, the algae harvested is capable of further being used in applications such foods, cosmetics, or biofuels.

Furthermore, the use of the automated algae culture apparatus in the invention includes applications in reducing energy consumption and carbon emissions, purifying air, landscaping and lighting.

In order to make the above and other objects, features and advantages of the present invention more comprehensible, several embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic view showing an automated algae culture apparatus according to an embodiment of the invention.

FIGS. 2 to 7 are schematic views each showing an automated algae culture apparatus according to other embodiments of the invention.

FIG. 8 is a flowchart showing an automated algae culture method according to an embodiment of the invention.

FIG. 9 is a graph showing growth curves of Spirulina according to the first experimental embodiment of the invention.

FIG. 10 is a graph showing growth curves of Spirulina according to the second experimental embodiment of the invention.

FIG. 11 is a graph showing a calibration curve for concentrations of algae according to the first and second experimental embodiments of the invention.

### DESCRIPTION OF EMBODIMENTS

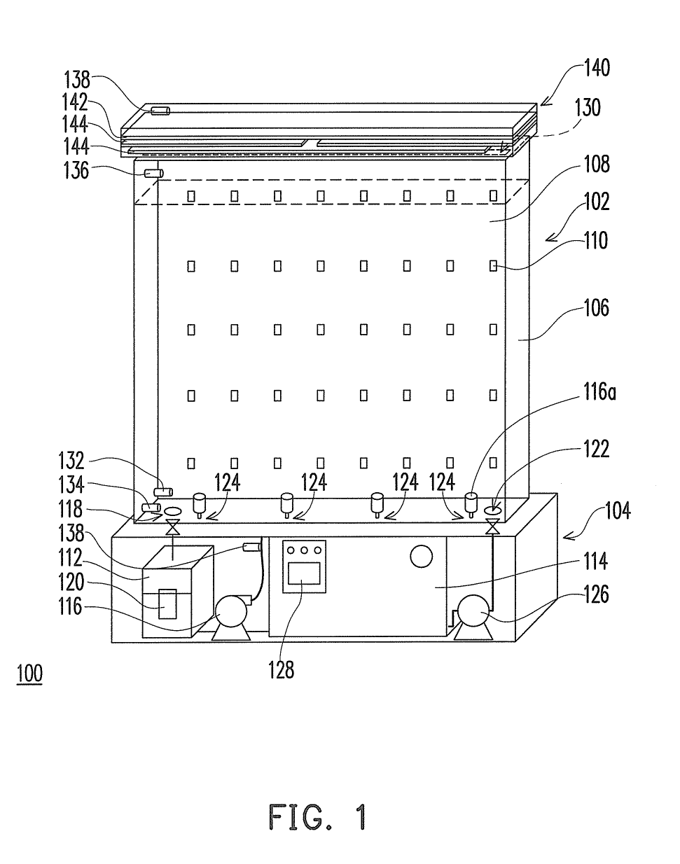

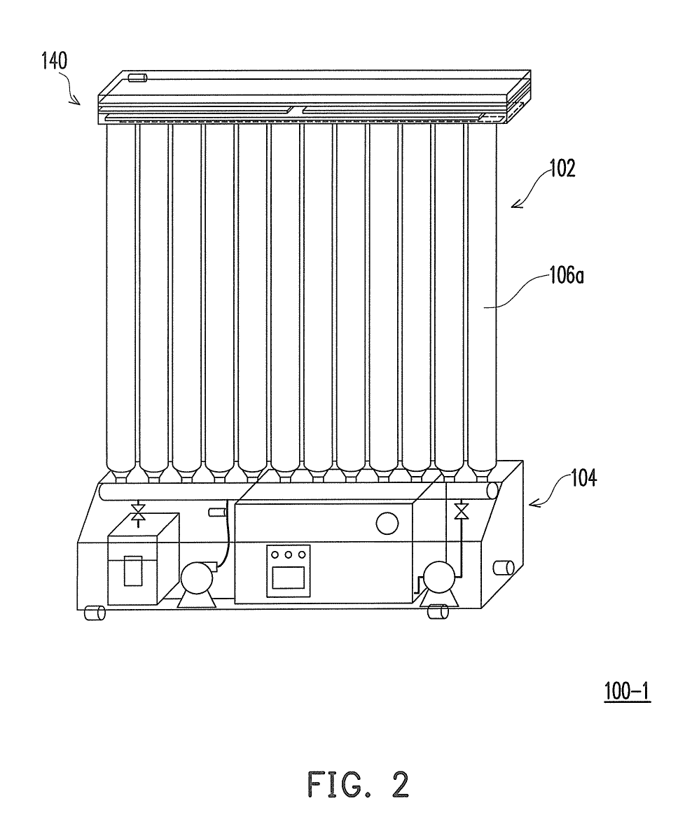

FIG. 1 is a schematic view showing an automated algae culture apparatus according to an embodiment of the invention. FIGS. 2 to 7 are schematic views each showing an automated algae culture apparatus according to other embodiments of the invention. In FIGS. 2 to 7, same modules as those in FIG. 1 may include same elements, wherein the same elements have same reference numerals and are not repeatedly described.

Referring to FIG. 1, an automated algae culture apparatus 100 includes at least one photosynthetic reactor module 102 and an auxiliary equipment module 104. In the automated algae culture apparatus 100, the auxiliary equipment module 104 may correspond to one or more photosynthetic reactor modules 102. According to the present embodiment, a situation in which one auxiliary equipment module 104 corresponds to one photosynthetic reactor module 102 is described.

The photosynthetic reactor module 102 includes a transparent container 106 used to contain an algae solution 108 which includes algae. The algae cultured in the present embodiment may be classified into freshwater algae or saltwater algae according to their aquatic environments for growth, or into microalgae or macroalgae according to their size. The algae cultured according to the present embodiment is, for example, green algae, blue green algae, or red algae. In addition to the algae planted therein, the algae solution 108 also includes a culture medium used for culturing the algae. A material of the transparent container 106 is, for example, glass, acrylic, or plastic. A manufacturing method thereof utilizes, for example, injection molding, vacuum molding, extrusion molding, or blow molding.

Referring to both FIGS. 1 and 2, the transparent container in the invention is not limited to the transparent container 106 shown as a thin-plate container in FIG. 1. Any container which is transparent and contains the algae solution 108 would suffice. For example, the transparent container may be a columnar container 106a of an automated algae culture apparatus 100-1 in FIG. 2.

In addition, still referring to FIG. 1, the photosynthetic reactor module 102 may further include an auxiliary light source 110 disposed on the transparent container 106, so as to illuminate the algae in the algae solution 108 for a sufficient time at night. The auxiliary light source 110 includes, for example, light-emitting diode (LED) crystalline grains or fluorescent lamps. According to the present embodiment, LED crystalline grains are used as an example for description. A power of the auxiliary light source 110 is, for example, from 30 W to 300 W.

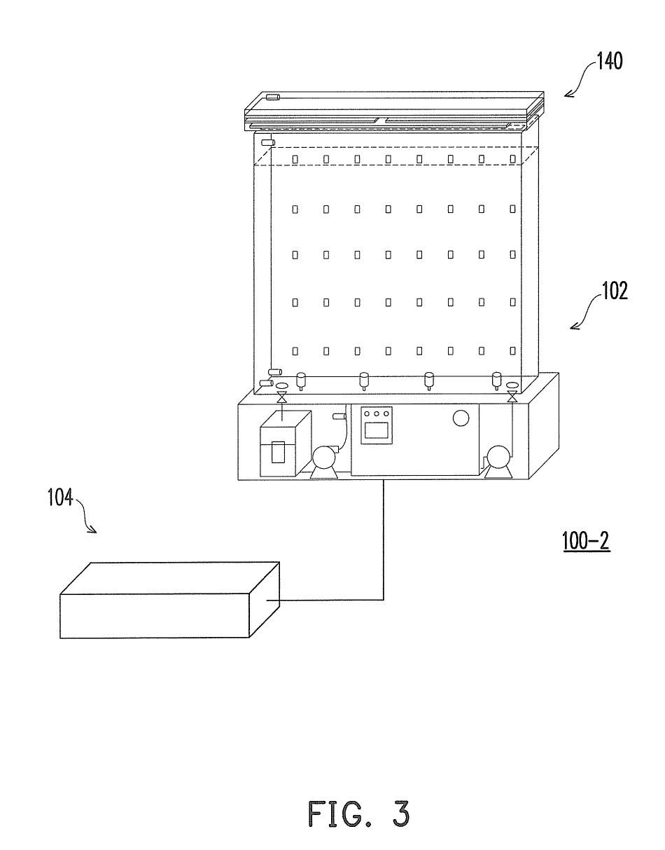

The auxiliary equipment module 104 includes a hydraulic filter 112, a buffer tank 114 and an aerator 116. Referring to both FIGS. 1 and 3, depending on whether the photosynthetic reactor module 102 and the auxiliary equipment module 104 are disposed in separation or in combination, the automated algae culture apparatus 100 is a separate apparatus or a combined apparatus. According to the embodiment in FIG. 1, the automated algae culture apparatus 100 is a combined apparatus in which the photosynthetic reactor module 102 and the auxiliary equipment module 104 are combined as one, but the invention is not limited thereto. According to the embodiment in FIG. 3, an automated algae culture apparatus 100-2 is a separate apparatus in which the photosynthetic reactor module 102 and the auxiliary equipment module 104 are separated from each other.

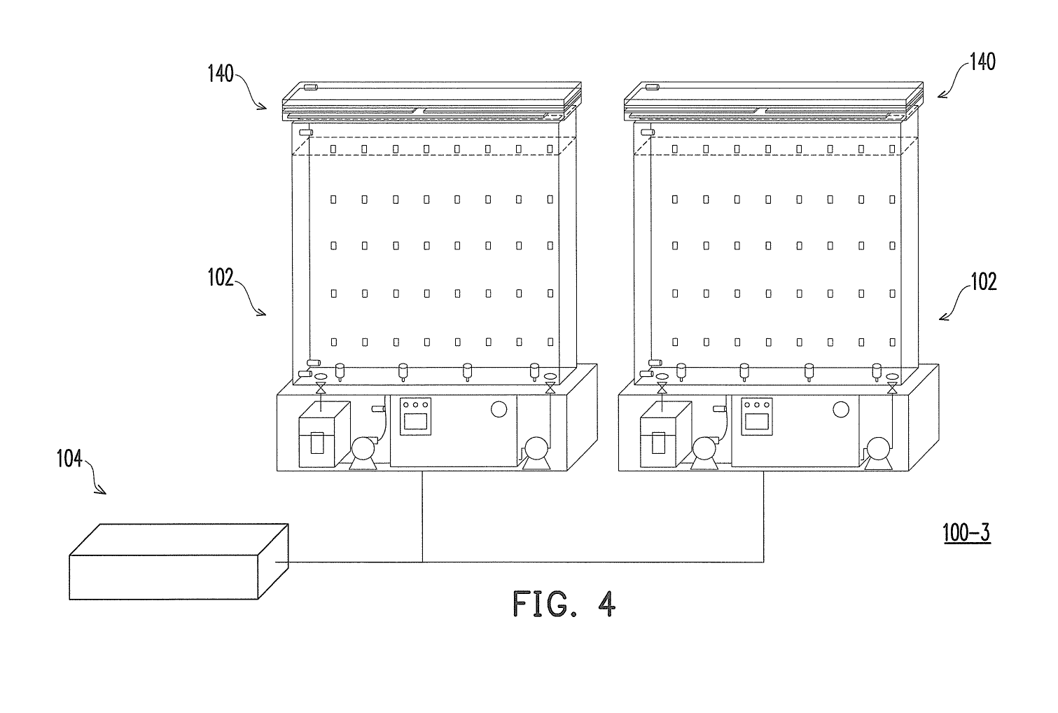

Referring to all FIGS. 1, 3 and 4, although in FIGS. 1 and 3, one auxiliary equipment module 104 corresponds to one photosynthetic reactor module 102, the invention is not limited thereto. For example, in an automated algae culture apparatus 100-3 in FIG. 4, one auxiliary equipment module 104 may correspond to a plurality of (two) photosynthetic reactor modules 102.

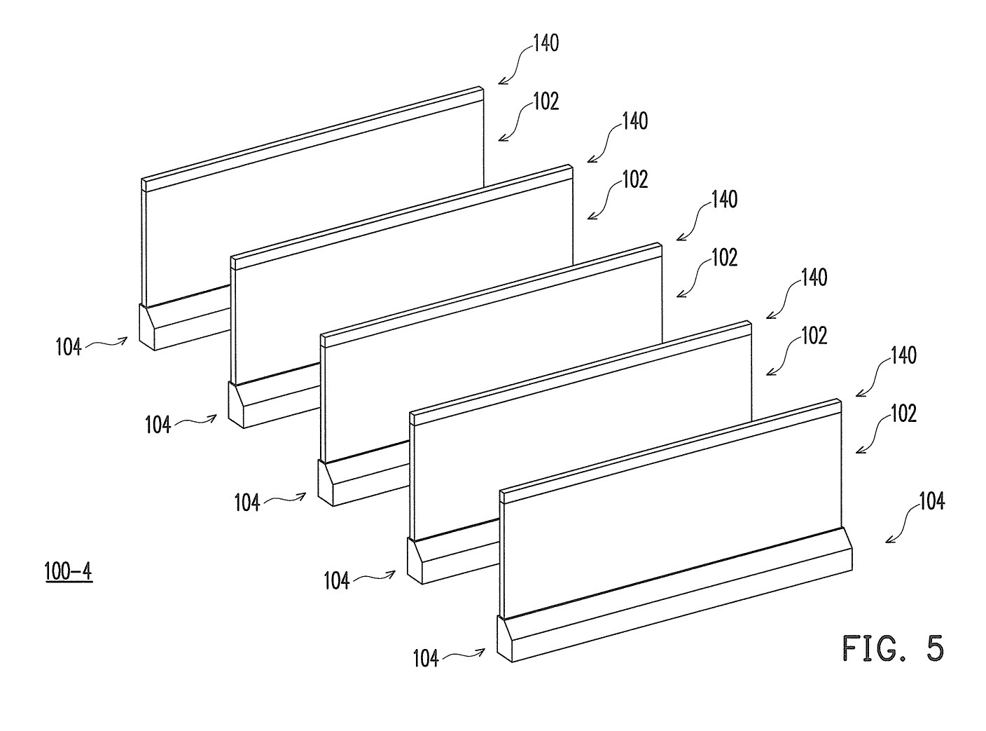

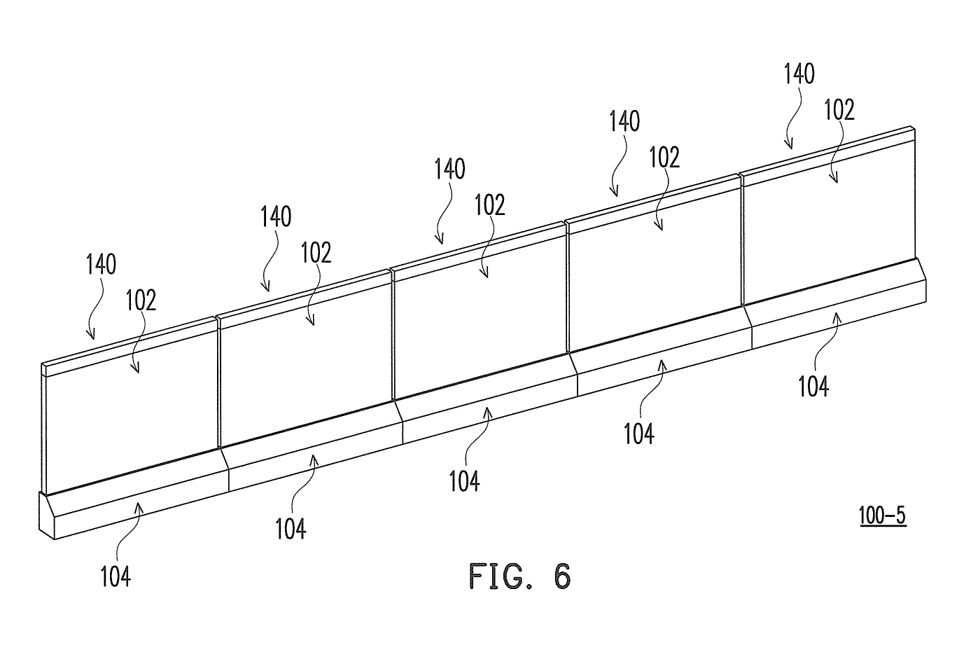

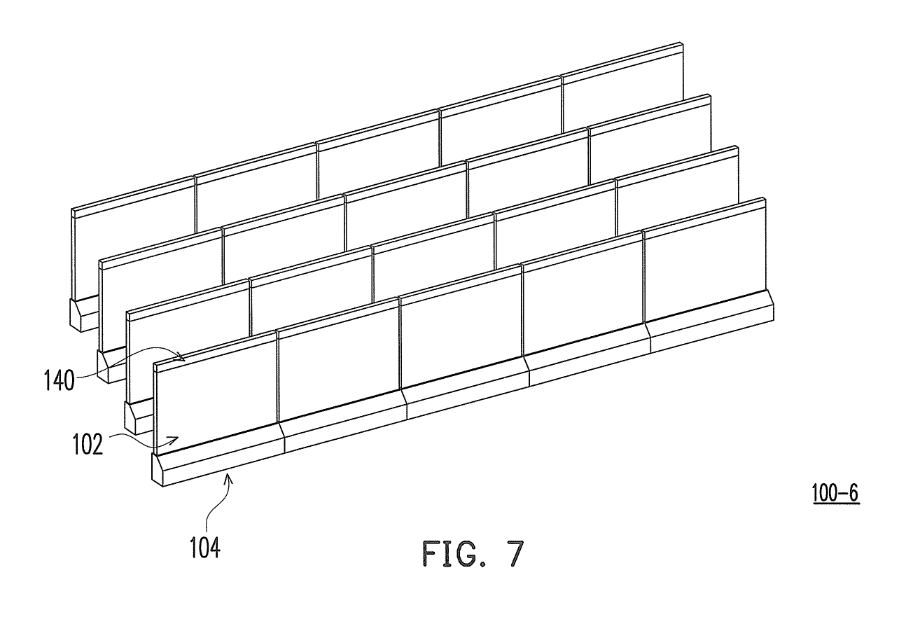

In addition, referring to all FIGS. 5 to 7, when the automated algae culture apparatus includes a plurality of photosynthetic reactor modules 102, persons having ordinary skills in the art may arrange the photosynthetic reactor modules 102 in a specific manner according to requirements. In an automated algae culture apparatus 100-4 in FIG. 5, an arrangement of the photosynthetic reactor modules 102 is, for example, a parallel arrangement. In an automated algae culture apparatus 100-5 in FIG. 6, an arrangement of the photosynthetic reactor modules 102 is, for example, a serial arrangement. In an automated algae culture apparatus 100-6 in FIG. 7, an arrangement of the photosynthetic reactor modules 102 is, for example, a matrix arrangement.

Equally, although the automated algae culture apparatuses 100-4, 100-5, and 100-6 in FIGS. 5 to 7 are combined apparatuses having components combined as a whole, the following describes a form in which one auxiliary equipment module 104 corresponds to one photosynthetic reactor module 102. However, persons having ordinary skills in the art may refer to the disclosure in FIGS. 3 and 4, so as to design the automated algae culture apparatuses 100-4, 100-5, and 100-6 in FIGS. 5 to 7 into separate devices and/or into fauns in which one auxiliary equipment module 104 corresponds to a plurality of photosynthetic reactor module 102.

Still referring to FIG. 1, the hydraulic filter 112 utilizes hydraulic pressure of the algae solution 108 in the transparent container 106 to filter the algae in the algae solution 108. In addition, the hydraulic filter 112 is communicated with a water outlet 118 of the transparent container 106 to filter the algae by gravity force, and does not require connection to a power source. The hydraulic filter 112 includes a filter bag or a filter screen 120. A material of the filter bag or filter screen 120 is, for example, non-woven cloth, polytetrafluoroethylene (TEFLON®), plastic, or synthetic fiber. A pore size of the filter bag or filter screen 120 is, for example, 0.5 μm to 200 μm, and may be selected depending on the type of algae cultured.

The buffer tank 114 is communicated with a water inlet 122 of the transparent container 106, so as to maintain a required fluid level of the algae solution 108 in the transparent container 106. By further being communicated with the hydraulic filter 112, the buffer tank 114 contains the filtrate filtered by the hydraulic filter 112. A ratio of the total capacities of the transparent container 106 to that of the buffer tank 114 is 10% to 80%. When the algae is harvested, generally 30% to 80% of the total algae solution is filtered, so that the remaining algae solution provides a sufficient initial concentration for culturing in a next stage. The filtrate may be reused for one week to half a year until changing and replenishing the water, depending on conditions of the different species of algae. If the volume of the buffer tank 114 is less than the volume of the filtered algae solution because of spatial limitations, the fluid level restoring pump 126 continuously replenishes the transparent container 106 with the filtrate. Since the algae solution in the transparent container 106 is continuously diluted by the filtrate, the filtering time is prolonged to increase the total filtered quantity, thereby obtaining a predetermined harvest quantity of dry algae.

The aerator 116 is communicated with an air inlet 124 of the transparent container 106 and pumps the air surrounding the aerator 116. An aerator head 116a of the aerator 116 pumps the air into the algae solution 108 in the transparent container 106, causing mixing and dissolution of carbon dioxide. Since carbon dioxide is heaver than oxygen and nitrogen, which are the main constituents of air, the aerator 116 may pump air from lower positions, so as to obtain higher concentrations of carbon dioxide. Alternative, the air inlet of the aerator 116 may be communicated with a vent of a factory, a kitchen, or an incinerator, or to foul air in a public space or household, so as to obtain higher concentrations of carbon dioxide.

In addition, the aerator 116 may be communicated with the hydraulic filter 112, so that the air pressure in the aerator 116 is utilized to pump the remaining algae solution 108 or filtrate into the buffer tank 114 and to blow dry the filtered algae, thereby forming dried algae.

In addition, the auxiliary equipment module 104 may further include a fluid level restoring pump 126 and a central controller 128. The fluid level restoring pump 126 is communicated with the transparent container 106 and the buffer tank 114 and is capable of injecting the filtrate in the buffer tank 114 and the nutrients added in the filtrate into the transparent container 106. The nutrients may be directly added into the buffer tank 114 as a liquid or solid powder. Alternatively nutrient powder may be added into a new filter bag or filter screen 120, so that every time the algae solution 108 is filtered, the nutrients are dissolved into the filtrate. The nutrients are then transferred into the transparent container 106 by the fluid level restoring pump 126.

The central controller 128 is used to monitor an environmental parameter of the algae solution 108. The environmental parameter is, for example, a temperature of the algae solution, a pH value of the algae solution, the fluid level of the algae solution, a first carbon dioxide concentration at the gas inlet 124 of the transparent container 106, a second carbon dioxide concentration at the gas outlet 130 of the transparent container 106, a lighting time, a harvest time, or any combination of the above parameters. The central controller 128 uses, for example, a temperature sensor 132, a pH value sensor 134, a fluid level sensor 136, and a carbon dioxide concentration sensor 138 of the auxiliary equipment module 104 to respectively measure the temperature of the algae solution, the pH value of the algae solution, the fluid level of the algae solution, the first carbon dioxide concentration at the gas inlet 124 of the transparent container 106, and the second carbon dioxide concentration at the gas outlet 130 of the transparent container 106. By measuring the first carbon dioxide concentration at the gas inlet 124 of the transparent container 106 and the second carbon dioxide concentration at the gas outlet 130 of the transparent container 106, the efficiency of carbon dioxide absorption is timely known. In addition, an optical density (OD) sensor (not shown) may be added in the transparent container to monitor the concentration of the algae solution in real time.

On the other hand, the automated algae culture apparatus 100 further includes an anti-fogging filter module 140 which covers the gas outlet 130 of the transparent container 106, so as to eliminate the water vapor and odor in the gas emitted from the photosynthetic reactor module 102. If the present system is disposed outdoors or is used for processing factory exhaust gas, the anti-fogging filter module 140 is optional.

The anti-fogging filtering module 140 includes a filter screen 142 and an anti-fogging partition 144. The filter screen 142 is disposed above the gas outlet 130. The filter screen 142 is, for example, an activated carbon filter screen. The anti-fogging partition 144 is disposed between the gas outlet 130 and the filter screen 142. When the automated algae culture apparatus 100 is equipped with the anti-fogging filter module 140, the carbon dioxide concentration sensor 138 used to sense the second carbon dioxide concentration at the gas outlet 130 of the transparent container 106 is disposed in the anti-fogging filter module 140 and above the filter screen 142.

In light of the above, since each of the automated algae culture apparatuses 100, 100-1, 100-2, 100-3, 100-4, 100-5, and 100-6 integrates several components into the auxiliary equipment module 104, a complete automated algae culture system is established by utilizing each of the automated algae culture apparatuses 100, 100-1, 100-2, 100-3, 100-4, 100-5, and 100-6, thereby miniaturizing, simplifying, and popularizing the algae culture system.

Therefore, the automated algae culture apparatuses 100, 100-1, 100-2, 100-3, 100-4, 100-5, and 100-6 are capable of being used at windowsides in an indoor space, so that when the sun is bright during daytime, sunlight is absorbed and heat insulated, thereby lowering the burden on the air conditioning system and purifying the air at the same time. Carbon dioxide is also absorbed, and oxygen generated during photosynthesis is emitted into the indoor space. At night, the auxiliary light sources in the automated algae culture apparatuses 100, 100-1, 100-2, 100-3, 100-4, 100-5, and 100-6 may be switched on, thereby maintaining photosynthesis and being used as a source for mild indoor lighting. Moreover, by culturing algae of different colors (such as green algae, blue green algae, and red algae), the automated algae culture apparatuses 100, 100-1, 100-2, 100-3, 100-4, 100-5, and 100-6 are capable of displaying different colors, thereby being great applications on indoor walls. Therefore, the automated algae culture apparatuses 100, 100-1, 100-2, 100-3, 100-4, 100-5, and 100-6 simultaneously have functions of purifying air, reducing energy consumption and carbon emissions, lighting, landscaping, and being decorative art.

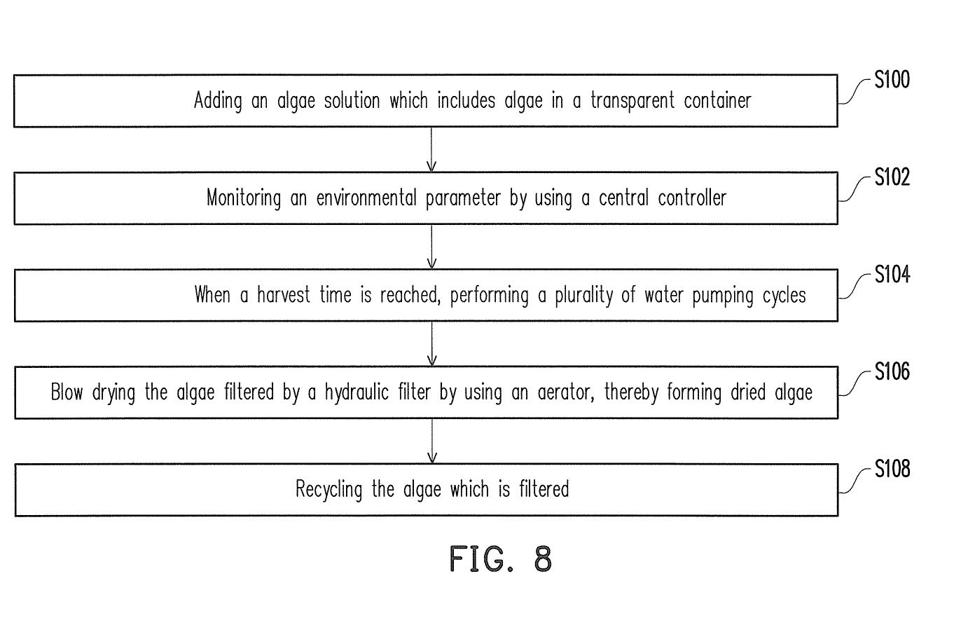

FIG. 8 is a flowchart showing an automated algae culture method according to an embodiment of the invention. In the automated algae culture method, the automated algae culture apparatus which is used is, for example, one of the automated algae culture apparatuses 100, 100-1, 100-2, 100-3, 100-4, 100-5, and 100-6 in FIGS. 1 to 7.

Referring to FIG. 8, a step 5100 is performed first, in which the algae solution including algae is added in the transparent container. The algae solution also includes the culture medium.

Next, a step S102 is performed. By monitoring the environmental parameter using the central controller, abnormalities in the automated algae culture apparatus are prevented. The environmental parameter is, for example, the temperature of the algae solution, the pH value of the algae solution, the fluid level of the algae solution, the first carbon dioxide concentration at the gas inlet of the transparent container, the second carbon dioxide concentration at the gas outlet of the transparent container, the lighting time, the harvest time, or any combination of the above parameters.

A step S104 is then performed. When the harvest time is reached, a plurality of water pumping cycles is performed. Each water pumping cycle includes following steps. First, the algae solution is automatically drained to the hydraulic filter, so that the algae and the filtrate are filtered, wherein the filtrate flows into the buffer tank. Next, the filtrate flows back from the buffer tank to the transparent container, until the predetermined fluid level of the algae solution is reached. Before the filtrate flows back from the buffer tank to the transparent container, nutrients may be further added into the filtrate. In step S104, for example, the central controller controls the time during which the valves are open, so that the algae solution is automatically drained to the hydraulic filter, and the filtrate flows back from the buffer tank to the transparent container by, for example, the fluid level restoring pump.

Next, a step S106 is selectively performed. By blow drying the algae filtered out by the hydraulic filter using the aerator, the dried algae is formed. Meanwhile, the remaining algae solution or filtrate is pumped into the buffer tank by the aerator.

Thereafter, in a step S108, the algae which is filtered is recycled. A method for recycling the algae is, for example, taking out the filter bag or filter screen from the hydraulic filter, so that the algae attached to the filter bag or filter screen is recycled.

As known from the above embodiments, the above automated algae culture apparatus is capable of automatically recycling the algae through the central controller and the hydraulic filter. In addition, the algae recycled using the above method is capable of being used in applications such as foods, cosmetics, or biofuels.

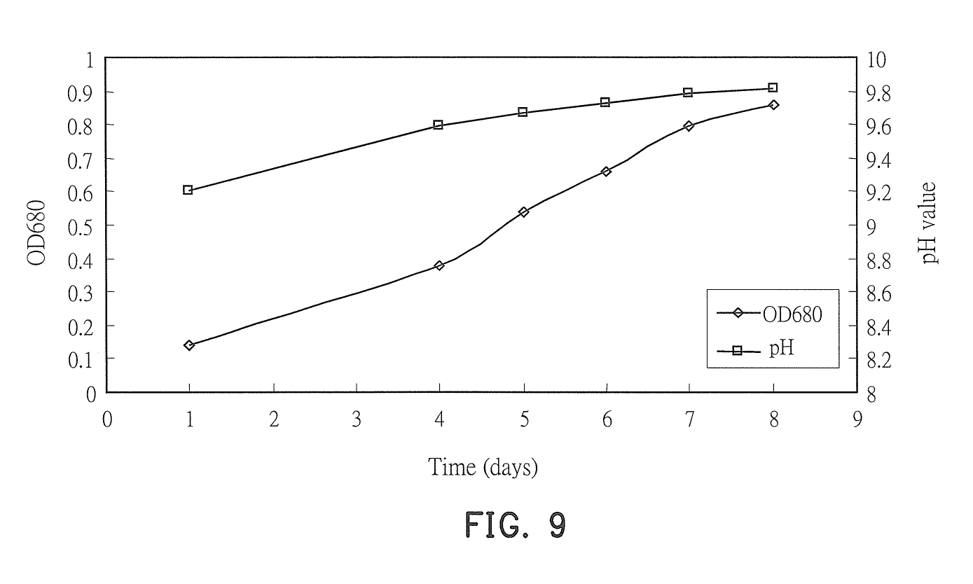

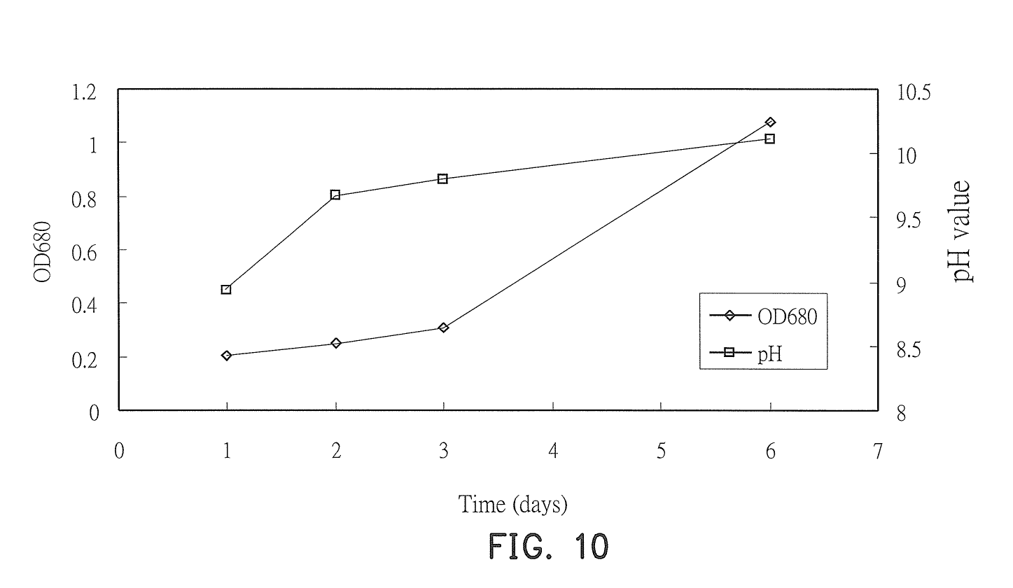

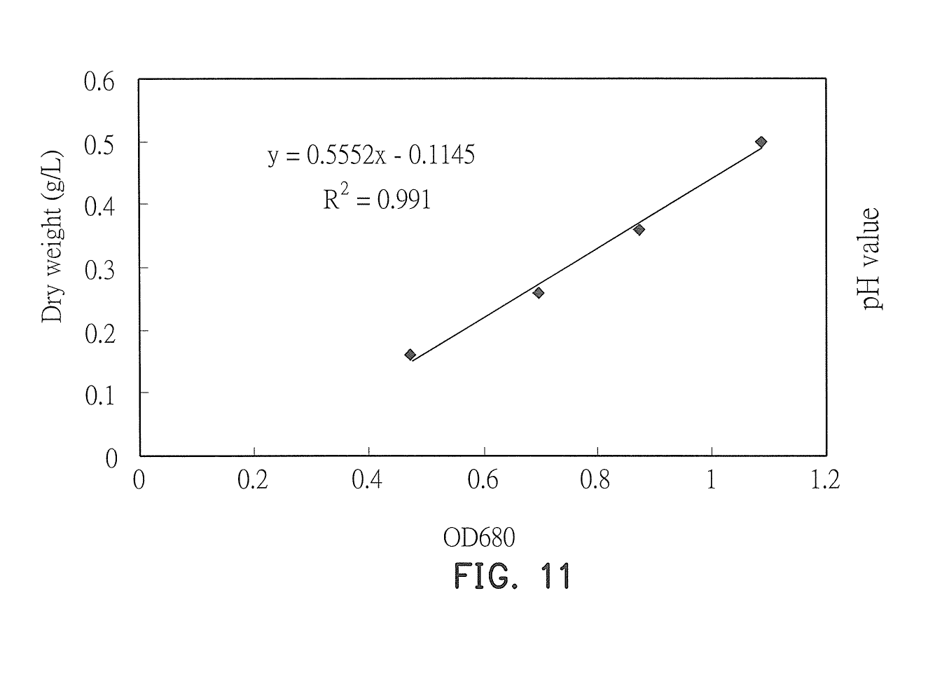

FIG. 9 is a graph showing growth curves of Spirulina according to the first experimental embodiment of the invention. FIG. 10 is a graph showing growth curves of Spirulina according to the second experimental embodiment of the invention. FIG. 11 is a graph showing a calibration curve for concentrations of algae according to the first and second experimental embodiments of the invention.

For operational conditions in the first experimental embodiment and the second experimental embodiment, the automated algae culture apparatus and method according to the above embodiments are used to culture the algae S. maxima, and the algae is aerated with air, whereas no additional carbon dioxide is supplemented.

Referring to all FIGS. 9 to 11, the concentrations of the algae in FIGS. 9 and 10 are represented by absorption values (at a wavelength of 680 nm). In FIG. 9, the absorption value for the initial concentration of the algae solution is 0.1, and in FIG. 10, the absorption value for the initial concentration of the algae solution is 0.2. During the 5 to 8 day period for culturing, each absorption value for the final concentrations may reach 5 to 8 times the absorption values for the initial concentrations. By applying the absorption values for the final concentrations in FIGS. 9 and 10 to the calibration curve representing absorption values and concentrations in FIG. 11, it is calculated that the final concentration of the algae solution in FIG. 9 is about 0.36 g/L, and the final concentration of the algae solution in FIG. 10 is about 0.5 g/L.

As known from the first and second experimental embodiments, by using the automated algae culture apparatuses in the above embodiments, the culture concentration is increased 5 to 8 times in one week. If high concentrations of carbon dioxide are added into the automated algae culture apparatus, the culture efficiency is further enhanced one to two times.

In summary, the above embodiments have at least the following advantages:

1. The automated algae culture apparatuses according to the above embodiments miniaturize and popularize the algae culture equipment.

2. The automated algae culture method is capable of automatically recycling algae, and the algae which is recycled is capable of being used in applications such foods, cosmetics, or biofuels.

3. The automated algae culture apparatuses in the above embodiments are capable of being used for reducing energy consumption and carbon emissions, purifying air, landscaping and lighting.

Although the present invention has been disclosed above by the embodiments, they are not intended to limit the present invention. Anybody skilled in the art can make some modifications and alterations without departing from the spirit and scope of the present invention. Therefore, the protecting range of the present invention falls in the appended claims.

What is claimed is:

1. An automated algae culture apparatus, comprising:
at least one photosynthetic reactor module, comprising a transparent container which is used to contain an algae solution comprising algae; and
an auxiliary equipment module, comprising:
a hydraulic filter, which is used to filter the algae in the algae solution, and is communicated with a water outlet of the transparent container to filter the algae by gravity force;
a buffer tank, communicated with a water inlet of the transparent container; and
an aerator, communicated with an air inlet of the transparent container.

2. The automated algae culture apparatus of claim 1, wherein the photosynthetic reactor module further comprises an auxiliary light source disposed on the transparent container.

3. The automated algae culture apparatus of claim 1, wherein the hydraulic filter comprises a filter bag or filter screen.

4. The automated algae culture apparatus of claim 1, wherein when the automated algae culture apparatus includes a plurality of photosynthetic reactor modules, an arrangement of the photosynthetic reactor modules includes a parallel arrangement, a serial arrangement, or a matrix arrangement.

5. The automated algae culture apparatus of claim 1, wherein the hydraulic filter is communicated with the buffer tank.

6. The automated algae culture apparatus of claim 1, wherein the hydraulic filter is communicated with the aerator.

7. The automated algae culture apparatus of claim 1, wherein the auxiliary equipment module further comprises a fluid level restoring pump communicated with the transparent container and the buffer tank.

8. The automated algae culture apparatus of claim 1, wherein the auxiliary equipment module further comprises a central controller which is used to monitor an environmental parameter of the algae solution.

9. The automated algae culture apparatus of claim 1, further comprising an anti-fogging filter module covering a gas outlet of the transparent container and comprising:
a filter screen, disposed above the gas outlet; and
an anti-fogging partition, disposed between the gas outlet and the filter screen.

10. The automated algae culture apparatus of claim 1, wherein the transparent container comprises a thin-plate container or a columnar container.

11. The automated algae culture apparatus of claim 1, wherein depending on whether the photosynthetic reactor module and the auxiliary equipment module are disposed in separation or in combination, the automated algae culture apparatus includes a separate apparatus or a combined apparatus.

12. The automated algae culture apparatus of claim 1, wherein the auxiliary equipment module corresponds to one or more photosynthetic reactor modules.
